(19) United States
(12) Patent Application Publication
(10) Pub. No.: US 2019/0031981 A1
(43) Pub. Date: Jan. 31, 2019

(54) CAPSULES

(71) Applicants: GIVAUDAN SA, Vernier (CH); L'UNIVERSITE PIERRE ET MARIE CURIE, Paris Cedex (FR); LE CENTRE NATIONAL DE LA RECHERCHE SCIENTIFIQUE, Paris Cedex (FR)

(72) Inventors: Stephane BONE, Estouteville Ecalles (FR); Cédric GEFFROY, Buxerolles (FR); Sandrine LE TIRILLY, Paris (FR); Patrick PERRIN, Paris (FR); Claire VAUTRIN, Pinsot (FR); Cècile MONTEUX, Paris (FR); Nadége PANTOUSTIER, Paris (FR)

(21) Appl. No.: 16/149,759

(22) Filed: Oct. 2, 2018

Related U.S. Application Data
(62) Division of application No. 14/432,101, filed on Mar. 27, 2015, now abandoned.

(30) Foreign Application Priority Data
Oct. 25, 2012 (EP) 12290370.1

(51) Int. Cl. C11D 3/50; A61Q 19/00; A61K 8/81; C11D 3/37; A61Q 15/00; A61Q 5/00; A61K 8/06; A61Q 5/12; A61Q 19/10; A61K 8/11; A61Q 13/00; A61Q 11/00; A61Q 5/02; B01J 13/22; A61K 8/02; A61Q 5/06

(52) U.S. Cl. CPC C11D 3/505; A61Q 19/00; A61Q 5/06; C11D 3/3757; A61Q 15/00; A61Q 5/00; A61K 8/06; A61Q 5/12; A61Q 19/10; A61K 8/11; A61Q 13/00; A61Q 11/00; A61K 8/8176; A61K 2800/56; A61Q 5/02; B01J 13/22; A61K 8/0245; A61K 8/8147

(57) ABSTRACT

Core-shell capsules comprising a fragrance-containing core and a shell formed around said core, wherein the shell is formed of polycarboxylic acid having a molecular weight of 1,000 to 10,000,000 Daltons

The present invention is concerned with encapsulation of beneficial agents, in particular fragrances.

Core-shell capsules are known in the art. They generally consist of a polymeric shell that is formed around a core containing a beneficial agent, such as a fragrance, and indeed any other ingredients that should be encapsulated.

Core-shell capsules are typically formed by forming droplets of core-forming material dispersed in an aqueous continuous phase. Monomers that should react to form a polymeric shell around the core droplets are contained in the droplets and/or the continuous phase. They can be made to react to form polymeric coatings around the droplets, which are subsequently consolidated (hardened) by crosslinking reactions. In this way, it is possible to form core-shell capsules comprising shells formed of aminoplast and resins, in particular thermoset resins, polyureas, polyurethanes, polyamides, gelatin, polyacrylics and the like.

Core-shell capsules are very versatile. The polymeric shell may have a barrier function, protecting the core contents from the environment external of the capsule, but it may also act as a means of modulating the release of fragrance at a desired rate of release. The nature and composition of the shell can influence capsule properties such as hardness, brittleness and porosity, and as such, affect the manner in which the beneficial agent can be released therefrom. A disadvantage of forming shells by in-situ polymerisation reactions is that the formed capsules can contain significant levels of unreacted monomers. Residues of monomers, for example, can be particularly undesirable in contact with skin, for instance in Personal Care applications. For this reason it is conventional to carry out numerous post-treatments on capsules to remove these residues after high conversion polymerisation, which is very difficult to achieve during interfacial polymerization. Post-treatment techniques may take the form of post-reacting with another molecule or post curing, volatilization, stream stripping, molecular trapping or extraction using a suitable solvent or ion-exchange resins. Unfortunately, these techniques are not particularly suitable when the capsules contain volatile benefit agents such as fragrances.

There remains a need to provide core-shell capsules, wherein the shell is based on polymers, which capsules contain very low levels of monomer residues.

Applicant has found that it is possible to form shells of pre-formed polymeric material based on polycarboxylic acids, around oil droplets comprising a beneficial agent, e.g. a fragrance. As the polymeric material is laid down pre-formed, capsules can be formed that contain extremely low levels of monomeric material heretofore unachievable using conventional in-situ polymerisation techniques.

The invention provides in a first aspect core-shell capsules comprising a fragrance-containing core and a shell formed around said core, wherein the shell is formed of polycarboxylic acid having a molecular weight of 1000 to 10 000 000 Daltons, more particularly 50 000 to 2 000 000 Daltons.

The capsules of the present invention are distinct from conventional polyacrylic capsules in that the shell is formed of pre-formed polymeric material as opposed to the conventional approach whereby monomers are reacted in situ to form the shell. In the latter case, the shell consists of a polymeric mass with no defined or measurable molecular weight.

There are several advantages attendant to the present invention. By employing pre-formed polymers to form the shell, one can exert great control over the shell's mechanical properties by selecting the desired molecular weight of the component polymers. Furthermore, the use of pre-formed polymeric materials substantially avoids the problem of residual monomeric contaminants being found in the capsules.

Accordingly, in another aspect of core-shell capsules comprising a fragrance-containing core and a shell formed around said core, wherein the shell is formed of polycarboxylic acid and wherein any carboxylic acid monomer is present in an amount of 100 ppm or less, more particularly 10 ppm or less, still more particularly 1 ppm or less.

Residual carboxylic acid can be measured by techniques known in the art, for example by GC analysis. Typically, a GC column may contain polydimethylsiloxane and the carrier gas may be helium. Monomeric residues can be extracted with a suitable solvent such as methanol or the like.

The polycarboxylic acid employed as a shell-forming material may be selected from the group consisting of polymethacrylic acid, polyacrylic acid, styrene-maleic acid copolymer, poly(itaconic acid), poly(monomethyl itaconate), poly(methyl methacrylate-co-methacrylic acid), poly(acrylic acid-co-maleic acid), poly(acrylic-co-methacrylic) acid, poly(methyl vinyl ether-alt-maleic acid), poly(styrene-co-methacrylic acid), poly(acrylic acid-co-maleic acid), poly(2-propylacrylic acid), poly(2-ethylacrylic acid), poly(styrene-alt-maleic acid), poly(ethylene-co-acrylic acid), poly(acrylamide-co-acrylic acid), poly(ethylene-co-methacrylic acid), poly(isobutylene-co-maleic acid), or poly(styrene-co-maleic acid); or acid anhydride polymers, which convert to acids under conditions of capsule formation such as ethylene-maleic anhydride copolymer, polyethylene-alt-maleic anhydride, poly(maleic anhydride-alt-1-octadecene), poly(isobutylene-alt-maleic anhydride), poly(styrene-co-maleic anhydride), poly(methyl vinyl ether-alt-maleic anhydride), poly(styrene-alt-maleic anhydride), poly[(isobutylene-alt-maleimide)-co-(isobutylene-alt-maleic anhydride)], or a polyanhydride of the Isobam type (supplied by Kuraray).

In an embodiment of the invention, the shell comprises one or more additional polymeric materials that is different from the polycarboxylic acid disclosed herein above.

In an embodiment of the present invention the shell consists of multiple layers of polycarboxylic acid, more particularly at least 2 layers, still more particularly 5 to 10 layers or more.

In an embodiment of the invention, the shell consists of multiple layers of polycarboxylic acid polymer material, and wherein each layer of polycarboxylic acid is interposed with a layer of said additional polymeric material.

In a more particular embodiment, the additional polymeric material forming said interposed layer or layers is selected for its property of being able to interact with contiguous polycarboxylic acid layers by forming either physical or chemical bonds therewith.

Still more particularly, the polymeric material forming said interposed layer or layers is able to interact with the adjacent polycarboxylic acid layer by forming hydrogen bonds.

The one or more additional polymeric materials referred to above may be selected from polyvinylpyrrolidone, polyvinyl alcohol, polymers, containing lactame groups like PVP or poly(N-vinyl caprolactame), polymers containing ether groups either in the backbone (Polyethylene oxide, poly propylene glycol) or as pendants (poly(vinyl methyl ether), acrylic type polymers such as polyacrylamide, poly(N-isopropylacrylamide) and poly(N,n-dimethylacrylamide), polymeric alcolohols such as PVA, poly(2-hydroxyethylacrylate) and poly(2-hydroxyethyl vinyl ether), other synthetic polymers such as poly(2-ethyl-2-oxazoline) and poly(N-acetyliminoethylene) and water soluble non ionic polysaccharides such as methylcellulose, hydroxyethylcellulose, hydroxypropylcellulose and hydroxypropylmethylcellulose.

Typical combinations include poly(methacrylic acid)/poly(vinyl pyrrolidone), poly(vinyl acetate)/ethylene-maleic anhydride copolymer and poly(vinyl alcohol)/ethylene-maleic anhydride copolymer.

By selecting the desired numbers of layers it is possible to closely control the thickness of the capsule shell. In a particular embodiment of the present invention the shell is from 5 to 150 nm thickness. By selecting the thickness of the shell as well as the nature of the pre-formed polymer material forming it, one is able to tailor the properties of the capsule shell in terms of porosity, robustness (to withstand the rigours of manufacture, compounding, transportation and storage) as well as friability (to permit the capsule to rupture in use at the desired time to release its fragrance).

The core-shell capsules are somewhat sensitive to high pH conditions. Indeed, at pH levels at 6.5 or above, the shells will begin to dissociate back into the polymeric material from which they were formed.

Accordingly, the invention provides in another of its aspects a method of stabilising said core-shell particles by storing them in a suitable medium at a pH of less than about 5, more particularly at a pH of about 2 to 4

In another aspect of the invention there is provided an aqueous slurry comprising core-shell capsules as herein described, wherein said slurry has a pH of less than about 5, more particularly 2 to 4.

The slurry may contain 5 to 50% by weight of core-shell capsules, more particularly 10 to 40%, still more particularly 20 to 30% by weight. Optionally, the slurry may contain a stabilizing polymer. It is common to employ stabilising polymers in slurries containing capsules and the skilled person would understand the common types of stabilising polymers that can be employed. A particular stabilising polymer is Keltrol. The stabilising polymer can be employed in amounts of about 0.05 to 5% by weight, more particularly 0.1 to 0.3%. The remaining mass of the slurry typically is comprised of water.

Having regard to the pH sensitivity of the core-shell capsules of the present invention, it is preferred if they are employed in applications in which the pH of the surrounding medium is 2 to 7. Core shell capsules of the present invention are particularly suitable for use in laundry applications including softeners, personal care and hair care applications including shampoo, conditioners, combing creams, leave on conditioners, styling cream, soaps, body creams; deodorants and anti-perspirants; household applications, fine fragrance and oral care.

The invention provides in another of its aspects a method of encapsulation of a beneficial agent, in particular a fragrance, comprising the steps of
(a) Dispersing oil droplets comprising said beneficial agent in an external phase comprising an aqueous medium having a pH of less than 6 to form a dispersed phase;
(b) Depositing around the dispersed phase droplets a shell comprising polycarboxylic acid and at least one additional polymeric material being different from the polycarboxylic acid wherein the polycarboxylic acid has a molecular weight of 1000 to 10 000 000 Daltons.

In a particular embodiment, there is provided a method of encapsulation of a beneficial agent, in particular a fragrance, comprising the steps of
(a) Dispersing oil droplets comprising said beneficial agent in an external phase comprising an aqueous medium having a pH of less than 6 to form a dispersed phase;
(b) Depositing around the dispersed phase droplets, layers comprising polycarboxylic acid,

wherein the polycarboxylic acid has a molecular weight of 1000 to 10 000 000 Daltons.

In a more particular embodiment there is provided a method of encapsulation of a beneficial agent, in particular a fragrance, comprising the steps of
(a) Dispersing oil droplets comprising said beneficial agent in an external phase comprising an aqueous medium having a pH of less than 6 to form a dispersed phase;
(b) Depositing around the dispersed phase droplets, alternate layers comprising polycarboxylic acid, and additional polymeric material being different from said polycarboxylic acid

wherein the polycarboxylic acid has a molecular weight of 1000 to 10 000 000 Daltons.

The technique of forming a shell around a core by the sequential deposition of layers of polymer around said core is known in the art. Previously, however, this technique has involved the alternate application of two polyelectrolytes to a sacrificial template core (typically silica) to build up a series of layers held together by electrostatic attraction. When the desired number of layers is attained, the core is then dissolved to give a hollow capsule, which may then be loaded with the desired beneficial agent. The technique offers the possibility of unprecedented control over release properties, and has been of great interest to the pharmaceutical industry (see, for example, Kozlovskaya et al, Chem. Mater. 2006, 18, 328-336).

In accordance with the present invention, it has been found possible to make core-shell capsules around liquid droplets containing a beneficial agent without the need of any kind of template or sacrificial core. Forming capsule shells around liquid droplets is more challenging than forming shells around solid cores because the former is a more mobile phase and ingredients can partition between the droplets and the external phase during the process of shell formation. Fragrances are particularly challenging to encapsulate in this way because their overall character depends on them consisting of complex, multi-component mixtures, which components have different physical properties and partition at different rates and to different extents.

Core-shell capsules formed according to the present invention are useful for encapsulating, among other useful beneficial agents, fragrance ingredients. The capsules can be formed with minimum loss of core material and maintenance of the hedonic character of the fragrance core created by the perfumer.

In forming a core-shell capsule by causing layers of polymeric material to be laid down on droplets of dispersed phase, once the initial two layers have been applied around said droplets, each succeeding layer need not be selected from the same materials as the initial two layers. The only requirement is that each succeeding layer is capable of interacting either physically or chemically with the layer adjacent it. This allows considerable versatility in how a capsule may be prepared in order to achieve a particular desired result.

The polymeric materials may be applied to droplets of a dispersed phase by introducing polymeric material to be deposited into the external phase surrounding the droplets under conditions permitting deposition of the polymeric material on the droplets. For example, the polymeric material can be introduced into the external phase in an amount of about 0.2 to 10% w/w in the external phase, more particularly 1 to 5% by weight, and at a pH of about 2 to 6. The amount of polymeric material introduced will depend upon its molecular weight (the higher the molecular weight, generally the lower amounts that will be employed in the external phase).

Different layers of polymeric material can be laid down sequentially, if after deposition of a layer of polymeric material is complete, the excess of that polymeric material is washed out of the external phase with water and a new (successive) polymeric material is introduced into the external phase. This process is repeated until the desired number of layers has been deposited. The process has the advantage that no particular proportions of polymer need be used, so no precise compositional limitations need be observed. The core-shell capsules may be formed in this manner using techniques including spray-coating or microfluidics.

Once the shell is formed, optionally, it may be crosslinked. This can be achieved by any convenient means. For example, polymers having amino or hydroxyl groups may be crosslinked by the addition of materials such as polyaldehyde, polyfunctional isocyanates or epoxides or any other suitable crosslinker.

Should it be desired, the shell thus formed can be used as a templating agent around which chemistry may be carried out to form aminoplast, polyurea, polyurethane, polyacrylic, inorganic microcapsules or hybrid capsules.

The disclosure is further described with reference to the following non-limiting examples.

### EXAMPLE 1

### Preparation of Fragrance-Containing Capsules

5 mL of a 1% (wt) poly (methacrylic acid) [PMAA] of 100,000 g/mol molecular weight aqueous solution is adjusted to pH to 3. To this solution is added 5 mL of a proprietary fragrance. The two solutions are kept in contact for 24 hours, and then emulsified using an Ultra-turrax™ blender at 24000 rpm for 2 minutes. The emulsion is added to a separating funnel and washed with water at pH 3 (10⁻³ M HCl) to give a 10% fragrance solution.

This solution is slowly mixed and then allowed to settle for 24 hours.

The lower aqueous phase is removed and washed four times with water at pH=3 to extract excess polymer. This washed phase is added to 1 (wt) % aqueous PVP (poly (vinyl pyrrolidone) of 40,000 g/mol molecular weight) solution at pH3 under gently stirring. This phase is allowed to settle for 24 h and it is then washed with water at pH 3 as described in the previous step. The settling, lower layer removal and washing steps are repeated.

The previous step is repeated with PMAA solution, and then again with PVP solution. This alternation is continued until 5 layers have been deposited.

The final fragrance-containing capsules have an average diameter of 31 μm and a wall thickness of about 14 nm.

### EXAMPLE 2

Preparation of Fragrance-Containing Capsules

The polymers used in this example are poly(acrylic acid) [PAA] of 50,000 g/mol molecular weight and poly(vinyl pyrrolidone) of 40,000 g/mol molecular weight.

5 mL of a 1% (wt) PAA aqueous solution is adjusted to pH to 3. To this solution is added 5 mL of a proprietary fragrance. The two solutions are kept in contact for 24 hours, and then emulsified using an Ultra-turrax™ blender at 24000 rpm for 2 minutes. The emulsion is added to a separating funnel and washed with water at pH 3 (10⁻³ M HCl) to give a 10% fragrance solution. This solution is slowly mixed and then allowed to settle for 24 hours.

The lower aqueous phase is removed and washed four times with water at pH=3 to extract excess polymer. This washed phase is added to 1 (wt) % aqueous PVP (poly (vinyl pyrrolidone) of 40,000 g/mol molecular weight) solution at pH3 under gently stirring. This phase is allowed to settle for 24 h and it is then washed with water at pH 3 as described in the previous step. The settling, lower layer removal and washing steps are repeated.

The previous step is repeated with PAA solution, and then again with PVP solution. This alternation is continued until 5 layers are attained.

The final fragrance-containing capsules have an average diameter of 31 μm and a wall thickness of about 18 nm

### EXAMPLE 3

### Polymer Recovery

1 g of capsule dispersion from Example 1 and Example 2 are introduced separately in 100 g of water at pH=8 to 9 under slow stirring. A white dispersion thus formed, first turns translucent and then transparent whilst an oily phase appears on the surface of the vessel when stirring is stopped.

The water phase is analysed by size exclusion chromatography using PL Aquagel-OH 8 μm (300×7.5 mm) and water at pH=7 with 0.2M NaNO3+0.01 M NaH2PO4 as eluent, PEG as calibration curve. Pure polymers used to manufacture capsule were analysed as well:

| Samples | Mw of Peak 1 (g/mol) | Mw of Peak 2 (g/mol) |
|---|---|---|
| Pure PMAA | 109,000 | — |
| Pure PVP | 45,000 | — |
| Pure PAA | 55,000 | — |
| Example 1 after recovery | 44,000 | 108,000 |
| Example 2 after recovery | 45,000 | 55,000 (overlapping) |

This demonstrates that polymers used in the formation of microcapsules are recovered after destruction of the capsules at a pH of between 8 and 9. This establishes that the capsule shell consists of discrete polymeric units of a defined molecular weight.

### EXAMPLE 4

### Preparation of a Flavour-Shifting Ice Cream Comprising 10-Layer PMAA/PVP Microcapsules of Example 1.

An ice cream is prepared by mixing the microcapsules containing a cherry flavour into an ice cream formulation at pH 5 containing a vanilla flavour.

The initial taste sensation is vanilla, quickly followed by cherry flavour after dissolution of capsules at higher pH in the mouth.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A method of encapsulation of a beneficial agent, comprising the steps of:
a) dispersing oil droplets comprising said beneficial agent in an external phase comprising an aqueous medium having a pH of less than about 6 to form a dispersed phase; and
b) causing the formation around the dispersed phase of a sequential series of as least two layers,
wherein the first layer is a poly(methacrylic acid) polymer having a molecular weight of from 1,000 to 10,000,000 Daltons, and the second layer is a poly(pyrrolidone) polymer, and wherein additional layers are layers alternating layers of a poly(methacrylic acid) polymer and a poly(pyrrolidone) polymer.

15. The method according to claim 14, wherein the beneficial agent is a fragrance composition.

16. The method of claim 14, wherein the aqueous medium has a pH of less from about 2 to about 6.

17. The method of claim 16, wherein the aqueous medium has a pH of less from about 2 to 4.

18. The method of claim 14 wherein the aqueous medium further comprises stabiliser and/or a buffer.

19. A consumer product composition having a pH of less than 6, selected from laundry cleaners and softeners, personal care and hair care products including shampoo, conditioners, combing creams, leave on conditioners, styling cream, soaps, body creams; deodorants and anti-perspirants; household applications, fine fragrance and oral care products, comprising core-shell capsules formed from the method of claim 14.

20. A method of stabilising core-shell capsules formed by the method of claim 14, the method comprising the step of storing them in a suitable medium at a pH of less than about 6.

21. The method of claim 14, wherein any carboxylic acid monomer contained in the capsules is present in an amount of 10 ppm or less.

22. The method of claim 21, wherein any carboxylic acid monomer contained in the capsules is present in an amount of 1 ppm or less.
